(19) United States
(12) Patent Application Publication
(10) Pub. No.: US 2023/0404373 A1
(43) Pub. Date: Dec. 21, 2023

(54) MAGNETIC CATHETER

(71) Applicant: 460Medical, Inc., Weston, MA (US)

(72) Inventors: Terrance J. Ransbury, Wake Forest, NC (US); Omar Amirana, Weston, MA (US)

(73) Assignee: 460Medical, Inc., Weston, MA (US)

(21) Appl. No.: 18/210,925

(22) Filed: Jun. 16, 2023

Related U.S. Application Data
(60) Provisional application No. 63/352,851, filed on Jun. 16, 2022.

(51) Int. Cl. A61B 1/00; A61B 1/07; A61B 1/06; A61B 1/04

(52) U.S. Cl. CPC A61B 1/00158; A61B 1/07; A61B 1/0653; A61B 1/00045; A61B 1/043; A61B 2018/00577

(57) ABSTRACT

Systems and methods for imaging tissue using a magnetic catheter are provided. In some embodiments, a catheter is provided that includes a catheter body having a proximal end, a distal end, and one or more lumens therebetween. One or more magnetic bodies are positioned along a length of the catheter body and are responsive to an applied magnetic field. At least one of the one or more magnetic bodies can be located at or near the distal end of the catheter body so that the distal end of the catheter can be manipulated with a magnetic field.

### RELATED APPLICATIONS

This application claims the benefit of and priority to U.S. Provisional Application No. 63/352,851, filed Jun. 16, 2022, the contents which is hereby incorporated herein by reference in its entirety.

### FIELD

The present disclosure generally relates to catheters, and more particularly ablation and visualization catheters.

### BACKGROUND

Ablation therapy is a minimally invasive procedure that applies energy to tissue to cause cell death. For example, ablation therapy can be used to remove or destroy abnormal tissue types (for example, tumors). Another example is the use of ablation therapy to treat atrial fibrillation (AF). Most episodes in patients with AF are known to be triggered by focal electrical activity originating from within muscle sleeves that extend into the Pulmonary Veins (PV). Atrial fibrillation may also be triggered by focal activity within the superior vena cava or other atrial structures, i.e. other cardiac tissue within the heart's conduction system. These focal triggers can also cause atrial tachycardia that is driven by reentrant electrical activity (or rotors), which may then fragment into a multitude of electrical wavelets that are characteristic of atrial fibrillation. Furthermore, prolonged AF can cause functional alterations in cardiac cell membranes and these changes further perpetuate atrial fibrillation. Physicians use a catheter to direct energy to either destroy focal triggers or to form electrical isolation lines isolating the triggers from the heart's remaining conduction system.

One issue with ablation therapy is the lack of visual feedback that often results in ineffective or incomplete ablation, and recurrence of the underlying medical condition. Therefore, there is a need for systems and methods that provide proper placement of a catheter for performing and visualizing ablation, and for forming and verifying proper lesions to improve outcomes and reduce costs.

### SUMMARY

According to some aspects of the present disclosure, a catheter comprising an optically-enabled, steerable magnetic catheter body having a proximal end, a distal end, and one or more lumens therebetween is provided. The catheter body includes regions of differing flexibility along a length thereof, with one or more magnetic bodies can be positioned along a length of the catheter body and being responsive to an applied magnetic field.

In some embodiments, a catheter is provided that includes a catheter body having a proximal end, a distal end, and one or more lumens therebetween. One or more magnetic bodies are positioned along a length of the catheter body and being responsive to an applied magnetic field, and at least one of the one or more magnetic bodies is located at or near the distal end of the catheter body so that the distal end of the catheter can be manipulated with a magnetic field.

In some embodiments, a distal-most magnetic body of the one or more magnetic bodies is configured to determine the position of the distal end of the catheter. In some embodiments, the proximal magnetic bodies are configured to prevent kinking of the catheter. In some embodiments, the distal end of the catheter body includes one or more openings for exchange of light energy between the distal end of the catheter body and tissue.

In some embodiments, the catheter body is flexible such that the magnetic bodies pull the catheter through a tortuous anatomy.

In some embodiments, spacing of the one or more magnetic bodies within a distal end of the catheter body is configured to modify how the catheter body is positioned and navigated. In some embodiments, each of the one or more magnetic bodies is configured to respond to the magnetic field in which it is placed to impact navigation of the catheter body.

In some embodiments, the catheter can further include one or more optical fibers extending to the distal end to deliver light energy to tissue.

In some embodiments, the distal end is configured to deliver ablation energy to tissue, the ablation energy being selected from a group consisting of radiofrequency (RF) energy, microwave energy, electrical energy, electromagnetic energy, cryoenergy, laser energy, ultrasound energy, acoustic energy, chemical energy, thermal energy and combinations thereof.

In some embodiments, a catheter is provided that includes an optically-enabled, steerable magnetic catheter body having a proximal end, a distal end, and one or more lumens therebetween, the catheter body having regions of differing flexibility along a length thereof, and one or more magnetic bodies positioned along a length of the catheter body and being responsive to an applied magnetic field. At least one of the one or more magnetic bodies is located at or near the distal end of the catheter body so that the distal end of the catheter can be manipulated with a magnetic field having practical strength.

In some embodiments, a distal-most magnetic body of the one or more magnetic bodies is configured to determine the position of the distal end of the catheter. In some embodiments, the proximal magnetic bodies are configured to prevent kinking of the catheter.

In some embodiments, the distal end of the catheter body includes one or more openings for exchange of light energy between the distal end of the catheter body and tissue.

In some embodiments, the catheter body is flexible such that the magnetic bodies pull the catheter through a tortuous anatomy. In some embodiments, spacing of the one or more magnetic bodies within a distal end of the catheter body is configured to modify how the catheter body is positioned and navigated. In some embodiments, each of the one or more magnetic bodies is configured to respond to the magnetic field in which it is placed to impact navigation of the catheter body.

In some embodiments, the catheter can further includes one or more optical fibers extending to the distal end to deliver light energy to tissue.

In some embodiments, the distal end is configured to deliver ablation energy to tissue, the ablation energy being selected from a group consisting of radiofrequency (RF) energy, microwave energy, electrical energy, electromagnetic energy, cryoenergy, laser energy, ultrasound energy, acoustic energy, chemical energy, thermal energy and combinations thereof.

In some embodiments, a method for visualizing ablated tissue is provided and includes advancing a catheter to a tissue in need of ablation, the catheter including a catheter body having a proximal end, a distal end, and one or more lumens therebetween; and one or more magnetic bodies positioned along a length of the catheter body and being responsive to an applied magnetic field, at least one of the one or more magnetic bodies located at or near the distal end of the catheter body so that the distal end of the catheter can be manipulated with a magnetic field; directing the light from the distal end of the catheter body to excite nicotinamide adenine dinucleotide hydrogen (NADH) in an area of the tissue including ablated tissue and non-ablated tissue; imaging the area of the tissue to detect NADH fluorescence of the area of the tissue; and producing a display of the imaged, illuminated tissue, the display illustrating the ablated tissue as having less fluorescence than non-ablated tissue.

In some embodiments, a method for visualizing ablated tissue is provided and includes advancing a catheter to a tissue in need of ablation, the catheter including an optically-enabled, steerable magnetic catheter body having a proximal end, a distal end, and one or more lumens therebetween, the catheter body having regions of differing flexibility along a length thereof; and one or more magnetic bodies positioned along a length of the catheter body and being responsive to an applied magnetic field, at least one of the one or more magnetic bodies located at or near the distal end of the catheter body so that the distal end of the catheter can be manipulated with a magnetic field; directing the light from the distal end of the catheter body to excite nicotinamide adenine dinucleotide hydrogen (NADH) in an area of the tissue including ablated tissue and non-ablated tissue; imaging the area of the tissue to detect NADH fluorescence of the area of the tissue; and producing a display of the imaged, illuminated tissue, the display illustrating the ablated tissue as having less fluorescence than non-ablated tissue.

### BRIEF DESCRIPTION OF THE DRAWINGS

The presently disclosed embodiments will be further explained with reference to the attached drawings, wherein like structures are referred to by like numerals throughout the several views. The drawings shown are not necessarily to scale, with emphasis instead generally being placed upon illustrating the principles of the presently disclosed embodiments.

FIG. 1 is an embodiment of an ablation visualization system of the present disclosure;

FIG. 2A is a diagram of a visualization system for use in connection with an ablation visualization system of the present disclosure;

FIG. 2B illustrates an exemplary computer system suitable for use in connection with the systems and methods of the present disclosure;

FIG. 3 illustrates an embodiment of a catheter of the present disclosure;

FIG. 4 illustrates an embodiment of a distal tip of the catheter shown in FIG. 3;

FIG. 5 illustrates an embodiment of a distal tip of a catheter;

FIG. 6A is an embodiment of an optically-enabled, variable stiffness, magnetic catheter;

FIG. 6B is a cross-sectional view of the catheter of FIG. 6A; and

FIG. 7 is an exemplary flow chart of a method of using a system of the present disclosure.

While the above-identified drawings set forth presently disclosed embodiments, other embodiments are also contemplated, as noted in the discussion. This disclosure presents illustrative embodiments by way of representation and not limitation. Numerous other modifications and embodiments can be devised by those skilled in the art which fall within the scope and spirit of the principles of the presently disclosed embodiments.

### DETAILED DESCRIPTION

The present disclosure generally relates to systems and methods for imaging tissue using nicotinamide adenine dinucleotide hydrogen (NADH) fluorescence (fNADH). By way of a non-limiting example, the present systems and methods can be used in connection with ablation therapy. In some embodiments, the catheter can be used to for applying energy, such as radiofrequency, pulsed field, modulated, laser or cryo ablation energy, to the body to form therapeutic lesions.

In general, the system can include a catheter with an optical system for exchanging light between tissue and the catheter. In some embodiments, the instant systems allow for direct visualization of the tissue's NADH fluorescence, or lack thereof, induced by ultraviolet (UV) excitation. The fluorescence signature returned from the tissue can be used to determine the presence or absence of ablation lesions in illuminated tissue as well as information about a lesion as it is forming during ablation. This optical tissue interrogation can be performed on various tissue types, including, without limitation, various cardiac tissues, endocardial tissue, epicardial tissue, myocardium tissue, valves, vascular structures, and fibrous and anatomical structures. The systems and methods of the present disclosure may be used to analyze tissue composition including, but not limited to the presence of collagen and elastin. However, the presently disclosed methods and systems may also be applicable for analyzing lesions in other tissue types, for example, skeletal muscle, liver, pancreas, brain, neural tissue, spleen, breast, uterus, cervical, prostate, bladder, esophagus, lung, pulmonary, arterial, blood clot or hematologic, gastrointestinal tract, adrenals, ovaries, testicles, genitourinary, kidney, and uterine. For example, the systems and methods described herein can also be used in urological applications, such as for ablation of kidney to treat kidney cancer. The lesions to be analyzed may be created by application of ablation energy during the ablation procedure. In some embodiments, existing lesions, created by ablation or by other means, may also be analyzed using methods and systems disclosed herein.

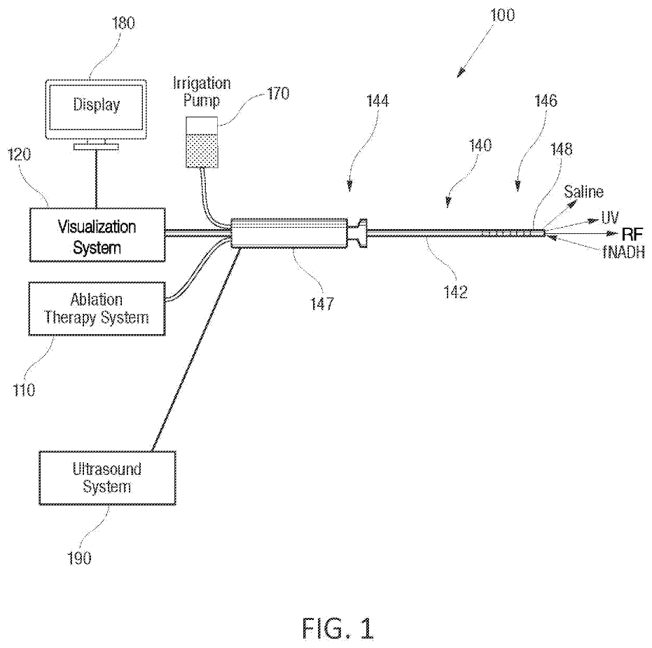

In reference to FIG. 1, the system for providing ablation therapy 100 may include an ablation therapy system 110, a visualization system 120, and a catheter 140. In some embodiments, the system 100 may also include an irrigation system 170. The system may also include a display 180, which can be a separate display or a part of the visualization system 120, as described below.

In some embodiments, the ablation therapy system 110 is designed to supply ablation energy to the catheter 140. The ablation therapy system 110 may include one or more energy sources that can generate radiofrequency (RF) energy, microwave energy, electrical energy (for example, pulsed field ablation), electroporation, electromagnetic energy, cryoenergy, laser energy, ultrasound energy, acoustic energy, chemical energy, thermal energy or any other type of energy that can be used to ablate tissue.

In some embodiments, the system includes an RF generator, an irrigation pump 170, an irrigated-tip ablation catheter 140, and the visualization system 120.

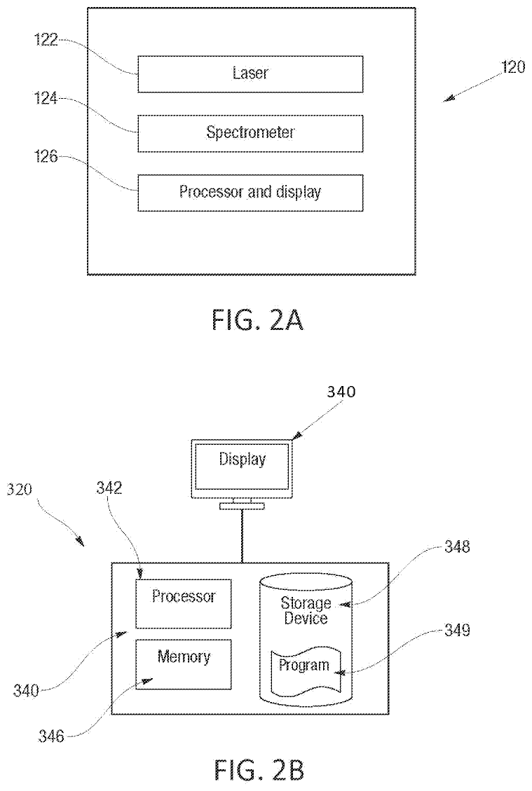

In reference to FIG. 2A, the visualization system 120 may include a light source 122, a light measuring instrument 124, and a computer system 126.

The computer system 126 can be programed to control various modules of the system 100, including, for example, control over the light source 122, control over the light measuring instrument 124, execution of application specific software, control over ultrasound, navigation and irrigation systems and similar operations. FIG. 2B shows, by way of example, a diagram of a typical processing architecture 320, which may be used in connection with the methods and systems of the present disclosure. A computer processing device 340 can be coupled to display 340AA for graphical output. Processor 342 can be a computer processor 342 capable of executing software. Typical examples can be computer processors (such as Intel® or AMD® processors), ASICs, microprocessors, and the like. Processor 342 can be coupled to memory 346, which can be typically a volatile RAM memory for storing instructions and data while processor 342 executes. Processor 342 may also be coupled to storage device 348, which can be a non-volatile storage medium, such as a hard drive, FLASH drive, tape drive, DVDROM, or similar device. Although not shown, computer processing device 340 typically includes various forms of input and output. The I/O may include network adapters, USB adapters, Bluetooth radios, mice, keyboards, touchpads, displays, touch screens, LEDs, vibration devices, speakers, microphones, sensors, or any other input or output device for use with computer processing device 340. Processor 342 may also be coupled to other types of computer-readable media, including, but not limited to, an electronic, optical, magnetic, or other storage or transmission device capable of providing a processor, such as the processor 342, with computer-readable instructions. Various other forms of computer-readable media can transmit or carry instructions to a computer, including a router, private or public network, or other transmission device or channel, both wired and wireless. The instructions may comprise code from any computer-programming language, including, for example, C, C++, C#, Visual Basic, Java, Python, Perl, and JavaScript.

Program 349 can be a computer program or computer readable code containing instructions and/or data and can be stored on storage device 348. The instructions may comprise code from any computer-programming language, including, for example, C, C++, C#, Visual Basic, Java, Python, Perl, and JavaScript. In a typical scenario, processor 342 may load some or all of the instructions and/or data of program 349 into memory 346 for execution. Program 349 can be any computer program or process including, but not limited to web browser, browser application, address registration process, application, or any other computer application or process. Program 349 may include various instructions and subroutines, which, when loaded into memory 346 and executed by processor 342 cause processor 342 to perform various operations, some or all of which may effectuate the methods for managing medical care disclosed herein. The program 349 may be stored on any type of non-transitory computer readable medium, such as, without limitation, hard drive, removable drive, CD, DVD or any other type of computer-readable media.

In some embodiments, the computer system may be programmed to perform the steps of the methods of the present disclosure and control various parts of the instant systems to perform necessary operation to achieve the methods of the present disclosure. In some embodiments, the processor may be programed to receive NADH fluorescence data from a tissue illuminated with UV light through the distal tip of the catheter, wherein the tissue is illuminated in a radial direction, an axial direction, or both; to determine from a level of NADH fluorescence in the illuminated tissue when the distal tip of the catheter is in contact with the tissue; and to cause (either automatically or by prompting the user) delivery of ablation energy to the tissue to form a lesion in the tissue upon determining that the distal tip is in contact with the tissue.

The processor may further be programmed for monitoring the level of NADH fluorescence during the delivering ablation energy to confirm that the distal tip remains in contact with the tissue. In some embodiments, monitoring the level of NADH fluorescence during the delivery of ablation energy may be utilized to determine stability of contact between the distal tip and the tissue. In some embodiments, ablation of the tissue may be stopped when the contact between the distal tip and the tissue is not stable. In some embodiments, the processor may further be programmed to collect a spectrum of fluorescence light returned from the illuminated tissue to distinguish tissue type.

In some embodiments, the light source 122 may have an output wavelength within the target fluorophore (NADH, in some embodiments) absorption range in order to induce fluorescence in healthy myocardial cells. In some embodiments, the light source 122 is a solid-state laser that can generate UV light to excite NADH fluorescence. In some embodiments, the wavelength may be about 355 nm or 355 nm+/−30 nm. In some embodiments, the light source 122 can be a UV laser. Laser-generated UV light may provide much more power for illumination and may be more efficiently coupled into a fiber-based illumination system, as is used in some embodiments of the catheter. In some embodiments, the instant system can use a laser with adjustable power up to 150 mW.

The wavelength range on the light source 122 may be bounded by the anatomy of interest, a user specifically choosing a wavelength that causes maximum NADH fluorescence without exciting excessive fluorescence of collagen, which exhibits an absorption peak at only slightly shorter wavelengths.

The wavelength range on the light source 122 may be bounded by the anatomy of interest, a user specifically choosing a wavelength that causes maximum NADH fluorescence without exciting excessive fluorescence of collagen, which exhibits an absorption peak at only slightly shorter wavelengths. In some embodiments, the light source 122 generates light having at least one wavelength between 250 nm and 450 nm. In some embodiments, the light source 122 generates light having at least one wavelength between 300 nm and 400 nm. In some embodiments, the light source 122 generates light having at least one wavelength between 330 nm and 385 nm. In some embodiments, the light source 122 generates light having at least one wavelength between 330 nm to 355 nm. In some embodiments, a narrow-band 355 nm source may be used. The output power of the light source 122 may be high enough to produce a recoverable tissue fluorescence signature, yet not so high as to induce cellular damage. The light source 122 may be coupled to an optical fiber to deliver light to and from the catheter 140, as will be described below.

In some embodiments, the light measuring instrument may monitor a level of the returned light having a wavelength between about 450 nm and 470 nm. In some embodiments, the monitored spectrum may be between 420 nm and 500 nm. In some embodiments, the monitored spectrum may be between 400 nm and 520 nm. Additionally or alternatively, a wider spectrum may be monitored, such as, by way of a non-limiting example, between 375 nm and 650 nm. In some embodiments, the NADH fluorescence spectrum and a wider spectrum may be displayed to user simultaneously. In some embodiments, the lesion may be created by ablation PFA energy. In some embodiments, the procedure may be started (by the processor or by prompting the user by the processor) when a NADH fluorescence peak is detected so it can be monitored throughout the procedure. As noted above, the processor may perform these methods in combination with other diagnostic methods, such as ultrasound monitoring.

In some embodiments, the systems of the present disclosure may utilize a spectrometer as the light measuring instrument 124. In some embodiments, the light measuring instrument 124 may comprise a camera connected to the computer system 126 for analysis and viewing of tissue fluorescence. In some embodiments, the camera may have high quantum efficiency for wavelengths corresponding to NADH fluorescence. One such camera is an Andor iXon DV860. The spectrometer 124 may be coupled to an imaging bundle that can be extended into the catheter 140 for visualization of tissue. In some embodiments, the imaging bundle for spectroscopy and the optical fiber for illumination may be combined. An optical bandpass filter of between 435 nm and 485 nm, in some embodiments, of 460 nm, may be inserted between the imaging bundle and the camera to block light outside of the NADH fluorescence emission band. In some embodiments, other optical bandpass filters may be inserted between the imaging bundle and the camera to block light outside of the NADH fluorescence emission band selected according to the peak fluorescence of the tissue being imaged.

In some embodiments, the light measuring instrument 124 may be a CCD (charge-coupled device) camera. In some embodiments, the spectrometer 124 may be selected so it is capable of collecting as many photons as possible and that contributes minimal noise to the image. Usually for fluorescence imaging of live cells, CCD cameras should have a quantum efficiency at about 460 nm of at least between 50-70%, indicating that 30-50% of photons will be disregarded. In some embodiments, the camera has quantum efficiency at 460 nm of about 90%. The camera may have a sample rate of 80 KHz. In some embodiments, the spectrometer 124 may have a readout noise of 8 e− (electrons) or less. In some embodiments, the spectrometer 124 has a minimum readout noise of 3 e−. Other light measuring instruments may be used in the systems and methods of the present disclosure.

The optical fiber 150 can deliver the gathered light to a long pass filter that blocks the reflected excitation wavelength of 355 nm but passes the fluoresced light that is emitted from the tissue at wavelengths above the cutoff of the filter. The filtered light from the tissue can then be captured and analyzed by a high-sensitivity spectrometer 124. The computer system 126 acquires the information from the spectrometer 124 and displays it to the physician. The computer 126 can also provide several additional functions including control over the light source 122, control over the spectrometer 124, and execution of application specific software.

In some embodiments, the digital image that is produced by analyzing the light data may be used to do the 2D and 3D reconstruction of the lesion, showing size, shape and any other characteristics necessary for analysis. In some embodiments, the image bundle may be connected to the spectrometer 124, which may generate a digital image of the lesion being examined from NADH fluorescence (fNADH), which can be displayed on the display 180. In some embodiment, these images can be displayed to the user in real time. The images can be analyzed by using software to obtain real-time details (e.g. intensity or radiated energy in a specific site of the image) to help the user to determine whether further intervention is necessary or desirable. In some embodiments, the NADH fluorescence may be conveyed directly to the computer system 126.

In some embodiments, the optical data acquired by the light measuring instrument can be analyzed to provide information about lesions during and after ablation including, but not limited to lesion depth and lesion size. In some embodiments, data from the light measuring instrument can be analyzed to determine if the catheter 140 is in contact with the myocardial surface and how much pressure is applied to the myocardial surface by the tip of the catheter. In some embodiments, data from the spectrometer 124 is analyzed to determine the presence of collagen or elastin in the tissue. In some embodiments, data from the light measuring instrument is analyzed and presented visually to the user via a graphical user interface in a way that provides the user with real-time feedback regarding lesion progression, lesion quality, myocardial contact, tissue collagen content, and tissue elastin content.

In some embodiments, the system 100 of the present disclosure may further include an ultrasound system 190. The catheter 140 may be equipped with ultrasound transducers in communication with the ultrasound system. In some embodiments, the ultrasound may show tissue depths, which in combination with the metabolic activity or the depth of lesion may be used to determine if a lesion is in fact transmural or not.

Referring back to FIG. 1, the catheter 140 includes a catheter body 142 having a proximal end 144 and a distal end 146. The catheter body 142 may be made of a biocompatible material and may be sufficiently flexible to enable steering and advancement of the catheter 140 to a site of ablation. In some embodiments, the catheter body 142 may have zones of variable stiffness. For example, the stiffness of the catheter 140 may increase from the proximal end 144 toward the distal end 146. In some embodiments, the stiffness of the catheter body 142 is selected to enable delivery of the catheter 140 to a desired cardiac location. In some embodiments, the catheter 140 can be a steerable, irrigated radiofrequency (RF) ablation catheter that can be delivered through a sheath to the endocardial space, and in the case of the heart's left side, via a standard transseptal procedure using common access tools. The catheter 140 may include a handle 147 at the proximal end 144. The handle 147 may be in communication with one or more lumens of the catheter to allow passage of instruments or materials through the catheter 140. In some embodiments, the handle 147 may include connections for the standard RF generator and irrigation system for therapy. In some embodiments, the catheter 140 may also include one or more adaptors configured to accommodate the optical fiber 150 for illumination and spectroscopy.

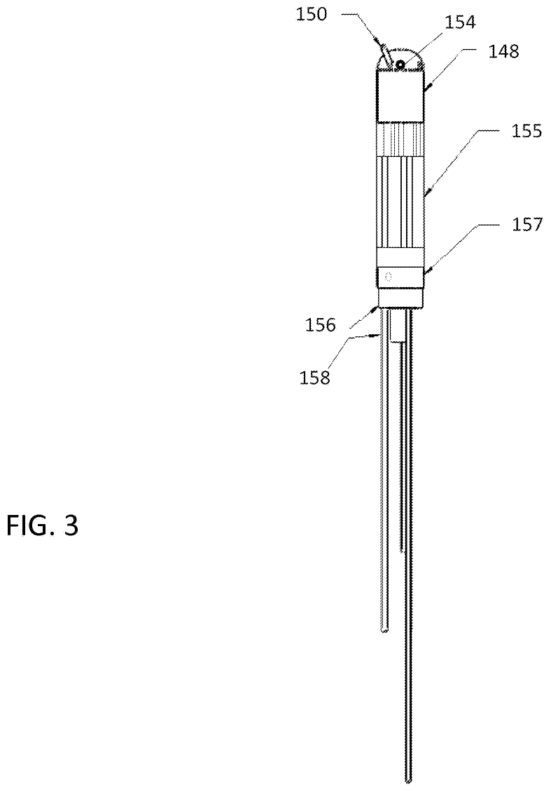

In reference to FIG. 3, at the distal end 146, the catheter 140 may include a distal tip 148. In some embodiments, the distal tip 148 may be configured to act as an electrode for diagnostic purposes, such as electrogram sensing, for therapeutic purposes, such as for emitting ablation energy, or both. In some embodiments where ablation energy is required, the distal tip 148 of the catheter 140 could serve as an ablation electrode or ablation element.

In the embodiments where RF energy is implemented, the wiring to couple the distal tip 148 to the RF energy source (external to the catheter) can be passed through a lumen of the catheter. The distal tip 148 may include a port in communication with the one or more lumens of the catheter. The distal tip 148 can be made of any biocompatible material. In some embodiments, if the distal tip 148 is configured to act as an electrode, the distal tip 148 can be made of metal, including, but not limited to, platinum, platinum-iridium, stainless steel, titanium or similar materials.

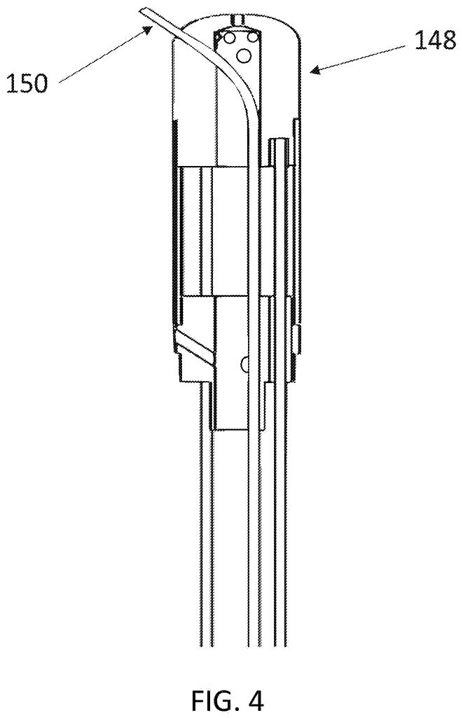

In reference to FIG. 3 and FIG. 4, an optical fiber or an imaging bundle may be passed from the visualization system 120, through the catheter body 142, and into an illumination cavity or compartment, defined by the distal tip 148. The distal tip 148 can be provided with one or more openings 154 for exchange of light energy between the illumination cavity and tissue. In some embodiments, even with multiple openings 154, the function of the distal tip 148 as an ablation electrode is not compromised. This light is delivered by one or more fibers 150 to the distal tip 148, where it illuminates the tissue in the proximity of the distal tip 148. This illumination light is either reflected or causes the tissue to fluoresce. The light reflected by and fluoresced from the tissue may be gathered by the optical fiber 150 within the distal tip 148 and carried back to the visualization system 120. In some embodiments, the same optical fiber or bundle of fibers 150 may be used to direct light to the tissue such that each fiber is coupled to each opening 154 to illuminate tissue outside the catheter 140 in one or more directions and to collect light from the tissue.

In some embodiments, the one or more openings 154 may be provided in any location on the walls of the distal tip 148. In some embodiments, the one or more openings 154 may be disposed circumferentially along the distal tip 148 around the entire circumference of the distal tip 148. In some embodiments, the one or more openings 154 may be disposed equidistantly from one another. The number of the openings may be determined by the desired angle of viewing coverage. For example, with 3 openings equally spaced, illumination and returned light occur at 120-degree increments (360 degrees divided by 3). In some embodiments, the one or more openings 154 may be provided in multiple rows along the walls of the distal tip 148. In some embodiments, the distal tip 148 may include 3 or 4 openings. In some embodiments, a single opening may be provided. In some embodiments, multiple openings 154 may be provided in the distal tip. In some embodiments, the distal tip 148 is provided with 3 side openings and 1 front opening. The one or more openings 154 may also serve as an irrigation port in connection with the irrigation system. In some embodiments light is only directed through some of the side openings 154. For example, in some embodiments there may exist 6 openings in the side wall, but light may be directed through only 3 of the openings, while the other openings may be used for irrigation. In some embodiments, the distal tip can include a flexible joint/irrigation line 155 for delivering fluid for irrigation. TC and RF wires 158 can pass through a ring 156 potted to allow for irrigation, and a first electrode/second fiber ring 157 can be oriented to the first ring 156.

To enable the light energy exchange between the distal tip of the catheter and tissue over multiple paths (axially and radially with respect to the longitudinal central axis of the catheter), one or more fibers 150 are positioned in the distal tip of the catheter. In some embodiments, each fiber is configured to be coupled to one of a plurality of openings in the distal tip 148, as shown in more detail in FIG. 4. While FIG. 4 illustrates a single fiber 150, it will be understood that this is for illustrative purposes only and that any number of fibers can be used. As shown in FIG. 4, the one or more optical fibers pass through the catheter to the distal tip thereof. A distal end of the fiber can be positioned such that it passes through one of the openings in the distal tip to direct light energy to tissue. It is possible that each fiber can be positioned through each opening, or there can be additional openings in the distal tip that can be used, for example, for irrigation or other functions.

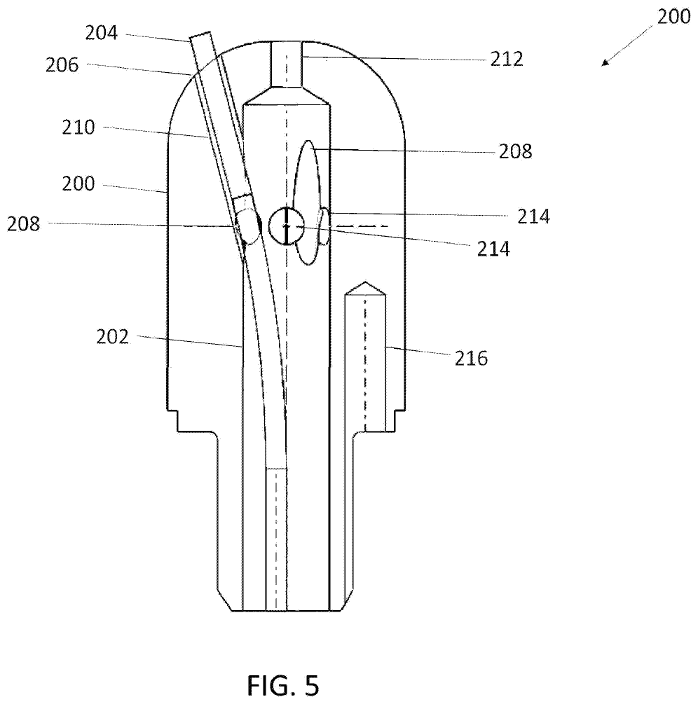

In some embodiments, the fibers are placed through the lumens in the tip with excess fiber. The fiber is then adhered to the tip typically using low or non-fluorescent epoxy. The excess fiber and any excess adhering compound can be trimmed at the outer surface of the tip and the fiber is then polished. The tip in FIG. 5 shows the excess fiber protruding from the tip before trimming. The fibers can be distributed around the tip in three dimensions to form as many light ports as any application would require. In some embodiments, full radial coverage with can be achieved with three radial ports separated at 120 degrees circumferentially around the catheter tip.

FIG. 5 illustrates an embodiment of a distal tip of a catheter for ablating tissue. As shown in FIG. 5, the distal tip 200 includes a lumen 202 for passing one or more optical fibers 204 and fluid through the catheter and into the distal tip 200. While the lumen 202 is shown as a single lumen, more than one lumen used such that there can be separate lumens for the optical fibers and the fluid.

The distal tip 200 can be provided with one or more openings 206 for exchange of light energy between the distal tip and tissue. This light is delivered by the one or more optical fibers 204 to the distal tip 148, where it illuminates the tissue in the proximity of the distal tip 148. Similar to the distal tip as described above, the illumination light is either reflected or causes the tissue to fluoresce, and the light reflected by and fluoresced from the tissue may be gathered by the optical fiber 204 and carried back to the visualization system 120. In some embodiments, the same optical fiber or bundle of fibers may be used to direct light to the tissue such that each fiber is coupled to each opening to illuminate tissue in one or more directions and to collect light from the tissue. A variety of configuration of openings can be positioned around the distal tip, including openings in a plurality of rows. The openings can be spaced randomly or evenly around the circumference of the distal tip. In some embodiments, there can be a combination of radial ports positioned around the circumference of the distal top, and forward-facing ports positioned at the distal end of the distal tip. For example, three radial openings can be positioned around the circumference of the distal tip, and the three openings can be evenly spaced with 120 degrees between each opening to provide full radial coverage. In some the openings can be arranged in multiple rows, with each row having openings positioned around the circumference of the distal tip. The three openings can be positioned in a single row or can be staggered along the length of the distal tip. In some embodiments, an opening 212 can be formed on the end of the distal top to allow for a forward-facing fiber to be used. The opening can be formed through the distal tip such that a channel is formed that extend between a distal end of the lumen and a distal end of the distal tip.

The shape of the distal tip can vary. As shown in FIG. 5, the distal tip is cylindrical in shape with substantially parallel walls. The distal end of the distal tip can be curved such that the distal end of the distal tip is slightly tapered. The ports or openings for the optical fibers can be positioned anywhere on the distal tip, but as shown in FIG. 5, in some embodiments, the radial ports or openings can be located on the curved portion of the distal tip, and the forward-facing port or opening is centered at the distal end of the distal tip.

Each opening 206 on the surface of the distal tip 200 can have a corresponding opening 208 in the lumen 202 such that an optical fiber can pass from the lumen, through one or more channels 210 formed in the distal tip, and to the openings 206 on the surface of the distal tip. In some embodiments, the distal tip 200 is a solid structure and the channels 210 can be formed by drilling through the distal tip from the lumen to the surface of the distal tip. The location, length, and angle of the channels formed between the lumen and the surface can vary depending on the location of the openings 208 in the lumen and the openings 206 in the surface of the distal tip. In some embodiments, the lumen can have additional openings to allow fluid to flow out of the lumen. As shown in FIG. 5, the lumen includes one or more fluid or irrigation ports that allow fluid to pass from the lumen to the tissue through channels formed through the distal tip. While the irrigation ports and corresponding channels and openings in the surface of the distal tip can be positioned anywhere along the distal tip, in some embodiments the irrigation ports are positioned between the openings 206.

A lumen 216 can also be include that is used to pass an energy-conducting element though the catheter and into the distal tip to connect the distal tip to an ablation system. The energy-conducting element can be used to delivery any type of ablation energy to the distal tip. For example, the energy-conduction element can be in the form of an electrode, such as an RF electrode. In some embodiments, the distal tip can be formed from a material that is configured to conduct energy, such as a metal, such that the distal tip can conduct the energy delivered through the lumen 216 by the energy-conducting element.

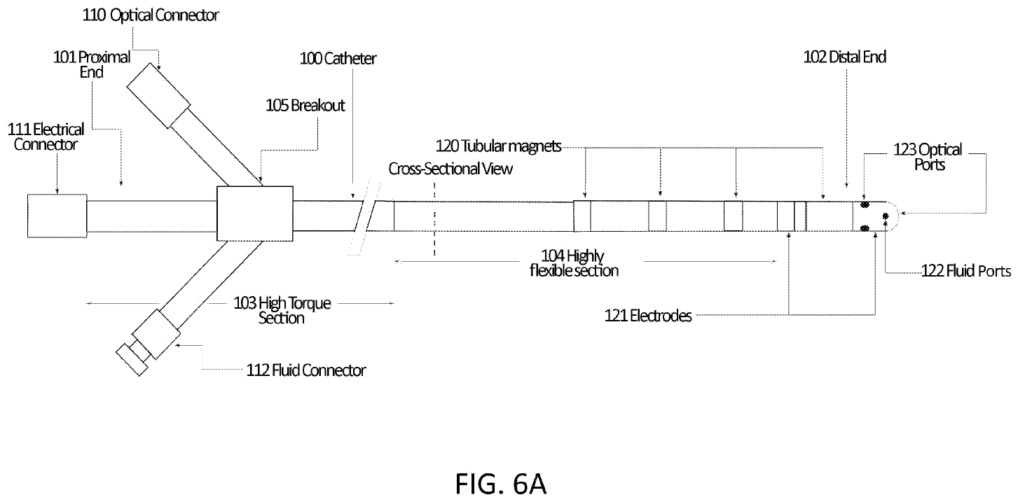

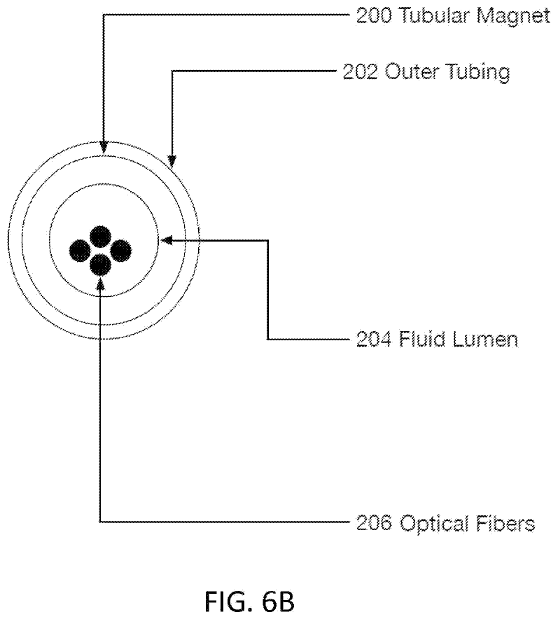

In some embodiments, an optically-enabled, steerable magnetic catheter having a proximal end, a distal end, and one or more lumens in between is provided. As shown in FIGS. 6A and 6B, the catheter has regions of differing flexibility along its length. There are one or more magnetic bodies, with one in particular located at or near the distal end, which are responsive to an applied magnetic field. The magnetic bodies are sized and the distal end portion of the catheter is designed so that the distal end of the catheter can be manipulated with a magnetic field of practical strength, eliminating the need for other mechanical deflection mechanisms, including a guidewire. In addition, the catheter includes one or more optical guides to transport light to and from the target anatomical site of interrogation.

In some embodiments, the catheter comprises a steerable magnetic catheter that is responsive to magnetic fields for the purposes of manipulating the catheter's location within the patient's body, specifically without a guidewire or other internal mechanical steering mechanisms.

Generally, the catheter has a proximal end and a distal end, and one or more lumens extending the length of the catheter. The catheter has regions of different flexibility along its length for optimal maneuverability within the body. As an example, in patients with congenital heart defects, access to specific areas of the heart may have limited or abnormal access paths to reach these locations. As such, the catheter may need to be floppy and flexible to navigate tortuosity to reach such locations. As opposed to a traditional ablation catheter which is steered from the proximal end to effect movement at the distal end which typically require stiffer catheter shafts for pushability and torque translation, a magnetically navigated catheter is configured to be “pulled” using the magnets located in the distal end of the catheter. Thus the shaft of the catheter can be more flexible from proximal end to distal end due to the use of the magnets to “pull” the catheter through tortuous anatomy. Adding varying degrees of flexibility along the catheter shaft can help navigation depending on anatomy and the desired locations to be accessed with the catheter tip for ablation.

In some embodiments, the proximal end has connectors for the electrical functions of the catheter, optical functions of the catheter, and/or an optional fluid connector. These connectors attach to instrumentation (not shown) specific to the medical procedure.

The lumens facilitate the optical fibers, electrical connections to any electrodes, electrical connection to sensors such as temperature sensors, and fluid to cool the tip electrode in case of thermal tissue ablation.

There are one or more magnetic bodies at the distal segment of the catheter. The bodies are responsive to an applied magnetic field, allowing the catheter to be shaped for navigation. The most distal magnetic body is such that it determines the position of the distal tip of the catheter while the proximal magnetic bodies primarily prevent kinking of the catheter. In some embodiments, the location of a distal magnet or the spacing of multiple magnets within the distal portion of the catheter can modify how the catheter is positioned and navigated which can aid in the procedure. Each magnet will respond to the magnetic field in which it is placed and hence can have impact on catheter navigation, access and stability.

The distal segment of the catheter is sufficiently flexible, and the magnets are sized such that the catheter can bend, in response to an applied magnetic field, at least 45 degrees and preferably 90 degrees with respect to the target anatomy.

With the objective of steering the catheter by manipulating the magnetic field, the distal end of the catheter can be very compliant as the embedded magnets in the catheter do the work to steer the catheter. The soft compliance of the distal end of the catheter is such that the material does not fight the influence of the magnetic pull. The compliant is selected to prevent the distal end from kinking due to overbending. Kinking can result in pinching off of the saline flow that cools the ablation tip during radiofrequency energy application.

When using any magnetic or non-magnetic catheter, high torque and column strength are required in the proximal (i.e., non-distal) length of the catheter for pushability and steerability. The pushability from column strength and rotational manipulation from the high torque section, allows the user (for example, a physician or robot) to deliver the distal end of the catheter to the target anatomic vicinity, where the magnets can take over in the fine placement of the distal tip. The term “high-torque” section of the catheter is relative to the very compliant distal end of the catheter. The torque must be enough to rotate the catheter without making the device too stiff for steering through the anatomy. Various methods to transmit torque within the catheter shaft can be used, for example including by the use of a braid or spiraled material such as metal or a stiffer polymer.

In some embodiments, a magnetic catheter is manipulated by two machines located proximal to the patient, while a physician can operate the machines away from the patient. The physician can be outside the sterile field, and more importantly for health reasons, can be outside of the radiation field due to the fluoroscopic system in the catheter lab. In some embodiments, the physician can manipulate the robot via a computer link to advance and retract the catheter. The physician can manipulate the distal end of the catheter via a computer that directs the magnetic system in the lab to create changes in the magnetic field that move the magnets within the catheter. The physician can also track the location of the catheter. In some embodiments, the catheter can be tracked using a fluoroscopic system. In some embodiments, the catheter can be traced using a commercially available mapping systems such as the CARTO™ system from JNJ or a similar system. The mapping systems track the movement of the catheter by triangulation from a sensor embedded in the catheter or by impedance from the tip electrode of the catheter to strategically spaced electrode patches on the patient or to neighboring catheters with navigation sensors.

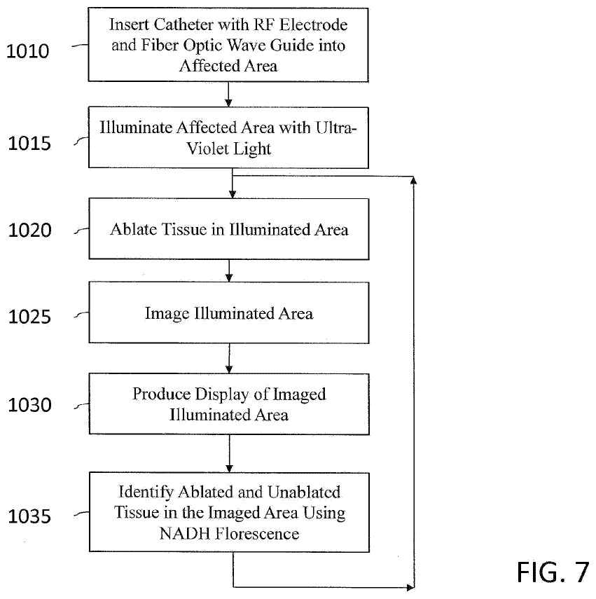

In reference to FIG. 7, operation of the systems 100 of the present disclosure is illustrated. Initially, the catheter 140 is inserted into the area of heart tissue affected by the atrial fibrillation, such as the pulmonary vein/left atrial junction or another area of the heart (step 1010). Blood may be removed from the visual field, for example, by irrigation. The affected area may be illuminated by ultra-violet light reflected from the light directing member 160 (step 1015). Tissue in the illuminated area may be ablated (step 1020), either before, after, or during illumination. Either point-to-point RF ablation or cryoablation or laser or other known ablation procedures may be employed using the systems of the present disclosure.

Still referring to FIG. 7, the illuminated area may be imaged with the light directing member receiving the light from the tissue and directing such light to the optical fiber, which can then pass the light to the spectrometer (step 1025). In some embodiments, the methods of the present disclosure rely on imaging of the fluorescence emission of NADH, which is a reduced form of nicotinamide adenine dinucleotide (NAD+). NAD+ is a coenzyme that plays important roles in the aerobic metabolic redox reactions of all living cells. It acts as an oxidizing agent by accepting electrons from the citric acid cycle (tricarboxylic acid cycle), which occurs in the mitochondrion. By this process, NAD+ is thus reduced to NADH. NADH and NAD+ are most abundant in the respiratory unit of the cell, the mitochondria, but are also present in the cytoplasm. NADH is an electron and proton donor in mitochondria to regulate the metabolism of the cell and to participate in many biological processes including DNA repair and transcription.

By measuring the UV-induced fluorescence of tissue, it is possible to learn about the biochemical state of the tissue. NADH fluorescence has been studied for its use in monitoring cell metabolic activities and cell death. Several studies in vitro and in vivo investigated the potential of using NADH fluorescence intensity as an intrinsic biomarker of cell death (either apoptosis or necrosis) monitoring. Once NADH is released from the mitochondria of damaged cells or converted to its oxidized form (NAD+), its fluorescence markedly declines, thus making it very useful in the differentiation of a healthy tissue from a damaged tissue. NADH can accumulate in the cell during ischemic states when oxygen is not available, increasing the fluorescent intensity. However, NADH presence disappears all together in the case of a dead cell. The following table summarizes the different states of relative intensity due to NADH fluorescence:

| | | Relative Changes of Auto- |
|---|---|---|
| Cellular State | NADH Presence | fluorescense intensity |
| Metabolically Active | Normal | Baseline |
| Metabolically Active but | Increased due | Increased |
| Impaired (Ischemia) | to Hypoxia | |
| Metabolically Inactive | Reduced | Reduced Attenuation |
| (Necrotic) | | |

Still referring to FIG. 7, while both NAD+ and NADH absorb UV light quite readily, NADH is autofluorescent in response to UV excitation whereas NAD+ is not. NADH has a UV excitation peak of about 340-360 nm and an emission peak of about 460 nm. In some embodiments, the methods of the present disclosure may employ excitation wavelengths between about 330 to about 370 nm. With the proper instrumentation, it is thus possible to image the emission wavelengths as a real-time measure of hypoxia as well as necrotic tissue within a region of interest. Furthermore, in some embodiments, a relative metric can be realized with a grayscale rendering proportionate to NADH fluorescence.

Under hypoxic conditions, the oxygen levels decline. The subsequent fNADH emission signal may increase in intensity indicating an excess of mitochondrial NADH. If hypoxia is left unchecked, full attenuation of the signal will ultimately occur as the affected cells along with their mitochondria die. High contrast in NADH levels may be used to identify the perimeter of terminally damaged ablated tissue.

To initiate fluorescence imaging, NADH may be excited by the UV light from the light source, such as a UV laser. NADH in the tissue specimen absorbs the excitation wavelengths of light and emits longer wavelengths of light. The emission light may be collected and passed back to the spectrometer, and a display of the imaged illuminated area may be produced on a display (step 1030), which is used to identify the ablated and unablated tissue in the imaged area based on the amount of NADH florescence (step 1035). For example, the sites of complete ablation may appear as completely dark area due to lack of fluorescence. Accordingly, the areas of ablation may appear markedly darker when compared to the surrounding unablated myocardium, which has a lighter appearance. This feature may enhance the ability to detect the ablated areas by providing marked contrast to the healthy tissue and even more contrast at the border zone between ablated and healthy tissue. This border area is the edematous and ischemic tissue in which NADH fluorescence becomes bright white upon imaging. The border zone creates a halo appearance around the ablated central tissue.

The process may then be repeated by returning to the ablation step, if necessary, to ablate additional tissue. It should be recognized that although FIG. 7 illustrates the steps being performed sequentially, many of the steps may be performed simultaneously or nearly simultaneously, or in a different order than shown in FIG. 7. For example, the ablation, imaging and display can occur at the same time, and the identification of the ablated and unablated tissue can occur while ablating the tissue.

In some embodiments, the system of the present disclosure comprises a catheter, a light source, and a light measuring instrument. In some embodiments, the system further comprises an optical detection system having an optical detection fiber, the optical detection system being independent or immune from electrical or RF energy noise. In some embodiments, the optical detection fiber does not conduct electrically and an RF energy does not produce electromagnetic energy in a range of interest to the system.

In some embodiments, the system is adapted to optically interrogate a catheter environment in a biologic system. In some embodiments, the system is adapted to optically interrogate in real-time, via an NADH fluorescence, the catheter environment to determine or assess one or more of a complete or a partial immersion of an electrode in a blood pool. For example, the optical system can detect, by inference, that the catheter tip is completely or partially immersed in the blood pool. The reason for this is because unlike the tissue or vasculature that return a positive optical signature, the blood completely absorbs the illumination light at this wavelength and thus returns a null optical signature. This feature of complete absorption provides optical isolation and therefore noise insulation. The instrument can use this situation for optical calibration and the elimination of stray optical signatures coming from the catheter itself. In addition, the system may be used for a qualitative and or a quantitative contact assessment between a catheter tip and a tissue, a qualitative and or a quantitative assessment of a catheter contact stability, an ablation lesion formation in real time, an ablation lesion progression monitoring, a determination of when to terminate a lesion, an identification of edematous zones which usually occur on a periphery of an ablation site and which can be associated with an incomplete ablation lesion, an ablation lesion depth, a cross-sectional area of the lesion, a temperature of the lesion, a recognition of steam formation or another physiologic parameter change to predict the onset of a steam pop, a formation of a char at a tip electrode during or after the ablation lesion formation, a detection of ischemia, a detection of a level of the ischemia, an ablation lesion assessment post lesion formation, an identification of edematous zones for re-ablation since edematous zones include myocardium that is electrically stunned, and a mapping of a location of previously ablated tissue by distinguishing metabolically active tissue from metabolically inactive tissue

In some embodiments, the system is adapted to optically interrogate a tissue parameter of an NADH fluorescence (fNADH).

In some embodiments, the system is adapted to optically interrogate a tissue, wherein the system analyzes parameters including a metabolic state of the tissue as well as a tissue composition of the tissue.

In some embodiments, the system is adapted to illuminate a tissue with a wavelength wherein illuminating leads to several optical responses. In some embodiments the optical responses comprises a myocardium containing NADH fluorescing if it is in a healthy metabolic state. In some embodiments, other tissues, such as collagen or elastin, fluoresce at different wavelengths, and the system uses a measurement of this information to determine a composition (i.e. collagen or elastin) of the tissue in contact with the catheter. In some embodiments the composition comprises myocardium, muscle, and myocardial structures such as valves, vascular structures, and fibrous or anatomical components. In some embodiments the composition comprises collagen, elastin, and other fibrous or support structures.

In some embodiments, a catheter of the present disclosure comprises a catheter body, a tip electrode, and one or more sensing electrodes. In some embodiments the catheter further comprises one or more zones of different flexibility, the zones of flexibility being in combination with a deflection mechanism adapted to allow a distal portion of the catheter to be bent for ease of navigation for a physician. In some embodiments, the zones of flexibility are located at the distal portion of the catheter, while a main body of the catheter is kept relatively stiff for pushability. In some embodiments, the main body of the catheter body is flexible so that the physician can use a robotic system for catheter navigation. In some embodiments the catheter is flexible and capable of being manipulated within a catheter sheath manually or robotically.

In some embodiments, the catheter further comprises a deflection mechanism adapted to deflect the catheter tip for navigation. In some embodiments the deflection mechanism comprises one or more pull wires that are manipulated by a catheter handle and which deflect the distal portion of the catheter in one or more directions or curve lengths. In some embodiments, the catheter further comprises a temperature sensor, the temperature sensor being integral to the distal tip of the electrode. In some embodiments the catheter further comprises one or more ultrasound transducers, the ultrasound transducers being located in the distal section of the catheter, and preferably in the tip of the distal electrode. The ultrasonic transducers are adapted to assess a tissue thickness either below or adjacent to the catheter tip. In some embodiments, the catheter comprises multiple transducers adapted to provide depth information covering a situation where the catheter tip is relatively perpendicular to a myocardium or relatively parallel to a myocardium.

In some embodiments the catheter further comprises an irrigation means for the purposes of flushing catheter openings with an irrigation fluid to clear the tip of blood, cooling a tissue-electrode interface, preventing a thrombus formation, and dispersing an RF energy to a greater zone of tissue, thus forming larger lesions than non-irrigated catheters. In some embodiments, the irrigating fluid is maintained within the catheter tip at a positive pressure relative to outside of the tip, and is adapted for continuous flushing of the openings.

In some embodiments, the catheter further comprises an electromagnetic location sensor adapted for locating and navigating the catheter. In some embodiments, the electromagnetic location sensor is adapted to locate the tip of the catheter in a navigation system of any one of several catheter manufacturers. The sensor picks up electromagnetic energy from a source location and computes location through triangulation or other means. In some embodiments the catheter comprises more than one transducer adapted to render a position of the catheter body and a curvature of the catheter body on a navigation system display.

In some embodiments, a catheter adapted to ablate tissue comprises a catheter body, and a tip electrode adapted to ablate a tissue. In some embodiments the catheter further comprises at least one optical waveguide adapted to deliver light energy to the tissue, and one or more optical waveguides adapted to receive light energy from the tissue. In some embodiments, the catheter further comprises a single optical waveguide adapted to deliver light energy to the tissue and receive light energy from the tissue.

In some embodiments, the catheter is adapted for an ablation energy, the ablation energy being one or more of RF energy, cryo energy, laser energy, chemical energy, electroporation energy, high intensity focused ultrasound or ultrasound energy, pulse field ablation energy, fluid modulated energy, and microwave energy.

In some embodiments, the tip of the catheter comprises a first electrode adapted for sensing electrical activity of the tissue, a second electrode adapted for transmitting or conducting ablation energy or chemical, a light directing member to direct a light in one or more directions simultaneously, one or more openings for the transmission and receiving of light energy, one or more openings for an irrigation fluid to flow from the tip, and one or more openings adapted for transmitting and receiving light as well as concomitantly flowing irrigation fluid from the tip. In some embodiments the tip of the catheter comprises an electrically conductive material, adapted to allow the first electrode to sense the electrical activity of the tissue in contact with the catheter. In some embodiments, the tip further comprises an electrode adapted for transmitting or conducting ablation energy or a chemical energy. In some embodiments, the tip is adapted to conduct RF energy to the adjacent tissue. In some embodiments, the tip comprises an optically transparent material allowing conduction of laser ablation energy to the adjacent tissue. In some embodiments, the tip comprises a plurality of holes adapted to transmit a chemical used to alter cells of the tissue or of a tissue in close proximity to the tip. In some embodiments, the openings for transmitting and receiving light are in the distal tip. In some embodiments, the tip comprises additional holes adapted to cool the tip with a fluid during an application of ablation energy.

In some embodiments, the tip further comprises at least one opening adapted to allow a directed light energy to illuminate the tissue, and to allow the light energy to return from the tissue to the catheter. In some embodiments, the tip comprises at least one opening in the distal tip for illuminating a tissue along a longitudinal axis of the catheter. In some embodiments, the light energy is directed in a manner that is dependent upon a light directing member having a central lumen allowing a portion of the light to be directed in a longitudinal direction. In some embodiments, the tip further comprises at least one opening in the distal tip for illuminating the tissue in a radial axis with respect to the catheter. In some embodiments, the tip is adapted to direct the light by splitting the primary light source into specific multiple beams using the light directing member.

In some embodiments, the primary light source is a laser, the laser adapted to send a light beam down an optical fiber to the light directing member, wherein the light beam is sent in one or more directions, including straight ahead relative to the tip, to make sure a structure adjacent to the catheter is illuminated. In some embodiments, a structure that is illuminated will transmit optical energy back to the catheter tip and to the light directing member, which in turn reflects the returned light back up the fiber to a spectrometer.

In some embodiments, the tip is configured to direct the light energy independent of any polishing of the interior of the illumination cavity. In some embodiments, the directing of light energy does not depend on the use of an interior wall of the illumination cavity.

In some embodiments, a catheter adapted to support fNADH comprising one or more ultrasound transducers. In some embodiments, the catheter is adapted to measure a wall thickness of an area of interest. In some embodiments, the catheter is adapted to assess a metabolic state of the tissue throughout the wall thickness. In some embodiments, the catheter further comprises ultrasonic transducers adapted to measure cardiac wall thickness and adapted to assess a metabolic state of the myocardium during an application of an RF energy. In some embodiments, the catheter is adapted to identify any metabolically active tissue for the purposes of identifying electrical gaps in lesions.

In some embodiments, the catheter comprises a light-directing component adapted to send light in one or more radial directions and axially simultaneously. In some embodiments, the catheter further comprises a separate or a modular component of the tip electrode, wherein an light directing member is integrated into the tip of the electrode during. In some embodiments, the light directing member has a centrally located lumen for light to pass in the axial direction.

In some embodiments, a catheter of the present disclosure comprises of a catheter body with the following components: a catheter with a distal tip positioned at a distal end of the catheter body, the distal tip defining a light chamber, the distal tip having one or more openings for exchange of light energy between the light chamber and tissue, and a the same catheter with a light directing member disposed within the light chamber, the light directing member being configured to direct the light energy to and from the tissue through the one or more openings in the distal tip. In some embodiments, the catheter comprises of one or more optical waveguides extending into the light chamber to deliver light to and from the light chamber. In some embodiments, the catheter has a light directing member and the one or more openings are configured to enable illumination of tissue in the radial and the axial directions. In some embodiments, the catheter has a distal tip that has a dome shaped front wall and straight side walls. In some embodiments, the catheter has one or more openings that are disposed along sidewalls of the distal tip. In some embodiments, the catheter has one or more openings that are disposed circumferentially along the distal tip. In some embodiments, the catheter has one or more openings that are provided in multiple rows along side walls of the distal tip. In some embodiments, the catheter has a distal tip that is comprised of a tissue ablation electrode. In some embodiments, the catheter has a light directing member that is configured to direct light radially through the one or more openings.

In some embodiments, the catheter has a light directing member that is rotatable with respect to the light chamber. In some embodiments, the catheter has a light directing member that is comprised of one or more through-holes and the distal tip is comprised of one or more openings disposed on a front wall of the distal tip to enable passage of light in longitudinal direction through the light directing member and the one or more openings of the front wall.

In some embodiments, there is provided a catheter for visualizing ablated tissue that includes a catheter body and a distal tip positioned at a distal end of the catheter body. The distal tip has one or more ports for an exchange of light energy between the distal tip and tissue. A lumen can extend through the catheter body and the distal tip and has one more openings corresponding to the one or more ports on the distal tip. One or more optical fibers are configured to extend through the lumen and the one or more openings to an outer surface of the distal tip through one or more ports in the distal tip such that the one or more optical fibers direct the light energy to and from the tissue through the one or more ports.

In some embodiments, the catheter can also include one or more channels configured to connect the one or more openings in the lumen to the one or more ports of the distal tip to direct the one or more optical fibers to the outer surface of the distal tip.

In some embodiments, the one or more ports are disposed circumferentially along the distal tip and are spaced apart from one another by equal distance. In some embodiments, one of the one or more ports is disposed at a distal end of the distal tip to direct light to tissue in front of the distal tip. In some embodiments, the one or more fibers are fixedly coupled to the one or more ports in the distal tip. In some embodiments, the light for illuminating the tissue has at least one wavelength between about 300 nm and about 400 nm.

In some embodiments, the distal tip is configured to deliver ablation energy to the tissue. The ablation energy is selected from a group consisting of radiofrequency (RF) energy, microwave energy, electrical energy, electromagnetic energy, cryoenergy, laser energy, ultrasound energy, acoustic energy, chemical energy, thermal energy and combinations thereof.

In some embodiments, the tissue is selected from a group consisting of skeletal muscle, liver, pancreas, brain, neural tissue, spleen, breast, uterus, cervical, prostate, bladder, esophagus, lung, pulmonary, arterial, blood clot or hematologic, gastrointestinal tract, adrenals, ovaries, testicles, genitourinary, and kidney.

A system for visualizing ablated tissue is provided that includes a catheter comprising a catheter body and a distal tip positioned at a distal end of the catheter body with the distal tip having one or more ports for exchange of light energy between the distal tip and tissue. A lumen extends through the catheter body and the distal tip and has one more openings corresponding to the one or more ports on the distal tip. One or more optical fibers are configured to extend through the lumen and the one or more openings to an outer surface of the distal tip through one or more ports in the distal tip such that the one or more optical fibers direct the light energy to and from the tissue through the one or more ports. The system also includes a light source, a light measuring instrument, and one or more optical fibers in communication with the light source and the light measuring instrument and extending through the catheter body into the distal tip. The one or more optical fibers are configured to pass light energy from the light source to the light directing member for illuminating tissue outside the distal tip and the one or more optical fibers are configured to relay light energy reflected from the tissue to the light measuring instrument.

In some embodiments, the one or more ports are disposed circumferentially along the distal tip and are spaced apart from one another by equal distance. In some embodiments, the system can also include an ultrasound transducer. In some embodiments, the light for illuminating the tissue has at least one wavelength between about 300 nm and about 400 nm. In some embodiments, the light measuring instrument is configured to detect returned light having a wavelength between about 450 nm and 470 nm is monitored.

In some embodiments, the system can include a source of ablation energy in communication with the distal tip to deliver ablation energy to the tissue. The ablation energy is selected from a group consisting of radiofrequency (RF) energy, microwave energy, electrical energy, electromagnetic energy, cryoenergy, laser energy, ultrasound energy, acoustic energy, chemical energy, thermal energy and combinations thereof.

According to some aspects of the present disclosure, there is provided a method for visualizing ablated tissue comprising advancing a catheter to a cardiac tissue in need of ablation. The catheter comprising a catheter body and a distal tip positioned at a distal end of the catheter body, with the distal tip having one or more ports for exchange of light energy between the distal tip and tissue. A lumen extends through the catheter body and the distal tip and having one more openings corresponding to the one or more ports on the distal tip. One or more optical fibers are configured to extend through the lumen and the one or more openings to an outer surface of the distal tip through one or more ports in the distal tip such that the one or more optical fibers direct the light energy to and from the tissue through the one or more ports. The method further includes illuminated the tissue through the one or more ports in the distal tip of the catheter to excite nicotinamide adenine dinucleotide hydrogen (NADH) in an area of the tissue including ablated cardiac tissue and non-ablated tissue, collecting light reflected from the tissue through the one or more openings and directing the collected light to a light measuring instrument, imaging the area of the tissue to detect NADH fluorescence of the area of the cardiac tissue, and producing a display of the imaged, illuminated tissue, the display illustrating the ablated cardiac tissue as having less fluorescence than non-ablated tissue.

In some embodiments, the method can further include ablating tissue with the distal tip prior to imaging the tissue. In some embodiments, the method can further include ablating additional non-ablated tissue identified by distinguishing between the ablated tissue and the non-ablated tissue based on the amount of fluorescence. In some embodiments, the tissue is selected from a group consisting of skeletal muscle, liver, pancreas, brain, neural tissue, spleen, breast, uterus, cervical, prostate, bladder, esophagus, lung, pulmonary, arterial, blood clot or hematologic, gastrointestinal tract, adrenals, ovaries, testicles, genitourinary, and kidney.

The foregoing disclosure has been set forth merely to illustrate various non-limiting embodiments of the present disclosure and is not intended to be limiting. Since modifications of the disclosed embodiments incorporating the spirit and substance of the disclosure may occur to persons skilled in the art, the presently disclosed embodiments should be construed to include everything within the scope of the appended claims and equivalents thereof.

What is claimed is:

1. A catheter, comprising:
a catheter body having a proximal end, a distal end, and one or more lumens therebetween; and
one or more magnetic bodies positioned along a length of the catheter body and being responsive to an applied magnetic field, at least one of the one or more magnetic bodies located at or near the distal end of the catheter body so that the distal end of the catheter can be manipulated with a magnetic field.

2. The catheter of claim 1, wherein a distal-most magnetic body of the one or more magnetic bodies is configured to determine the position of the distal end of the catheter.

3. The catheter of claim 1, wherein the proximal magnetic bodies are configured to prevent kinking of the catheter.

4. The catheter of claim 2, wherein the distal end of the catheter body includes one or more openings for exchange of light energy between the distal end of the catheter body and tissue.

5. The catheter of claim 1, wherein the catheter body is flexible such that the magnetic bodies pull the catheter through a tortuous anatomy.

6. The catheter of claim 1, wherein spacing of the one or more magnetic bodies within a distal end of the catheter body is configured to modify how the catheter body is positioned and navigated.

7. The catheter of claim 1, where each of the one or more magnetic bodies is configured to respond to the magnetic field in which it is placed to impact navigation of the catheter body.

8. The catheter of claim 1, further comprising one or more optical fibers extending to the distal end to deliver light energy to tissue.

9. The catheter of claim 1, wherein the distal end is configured to deliver ablation energy to tissue, the ablation energy being selected from a group consisting of radiofrequency (RF) energy, microwave energy, electrical energy, electromagnetic energy, cryoenergy, laser energy, ultrasound energy, acoustic energy, chemical energy, thermal energy and combinations thereof.

10. A catheter, comprising:
an optically-enabled, steerable magnetic catheter body having a proximal end, a distal end, and one or more lumens therebetween, the catheter body having regions of differing flexibility along a length thereof; and
one or more magnetic bodies positioned along a length of the catheter body and being responsive to an applied magnetic field, at least one of the one or more magnetic bodies located at or near the distal end of the catheter body so that the distal end of the catheter can be manipulated with a magnetic field having practical strength.

11. The catheter of claim 10, wherein a distal-most magnetic body of the one or more magnetic bodies is configured to determine the position of the distal end of the catheter.

12. The catheter of claim 10, wherein the proximal magnetic bodies are configured to prevent kinking of the catheter.

13. The catheter of claim 12, wherein the distal end of the catheter body includes one or more openings for exchange of light energy between the distal end of the catheter body and tissue.

14. The catheter of claim 10, wherein the catheter body is flexible such that the magnetic bodies pull the catheter through a tortuous anatomy.

15. The catheter of claim 10, wherein spacing of the one or more magnetic bodies within a distal end of the catheter body is configured to modify how the catheter body is positioned and navigated.

16. The catheter of claim 10, where each of the one or more magnetic bodies is configured to respond to the magnetic field in which it is placed to impact navigation of the catheter body.

17. The catheter of claim 10, further comprising one or more optical fibers extending to the distal end to deliver light energy to tissue.

18. The catheter of claim 10, wherein the distal end is configured to deliver ablation energy to tissue, the ablation energy being selected from a group consisting of radiofrequency (RF) energy, microwave energy, electrical energy, electromagnetic energy, cryoenergy, laser energy, ultrasound energy, acoustic energy, chemical energy, thermal energy and combinations thereof.

19. A method for visualizing ablated tissue, comprising:
advancing a catheter to a tissue in need of ablation, the catheter comprising a catheter body having a proximal end, a distal end, and one or more lumens therebetween; and one or more magnetic bodies positioned along a length of the catheter body and being responsive to an applied magnetic field, at least one of the one or more magnetic bodies located at or near the distal end of the catheter body so that the distal end of the catheter can be manipulated with a magnetic field;
directing the light from the distal end of the catheter body to excite nicotinamide adenine dinucleotide hydrogen (NADH) in an area of the tissue including ablated tissue and non-ablated tissue;
imaging the area of the tissue to detect NADH fluorescence of the area of the tissue; and
producing a display of the imaged, illuminated tissue, the display illustrating the ablated tissue as having less fluorescence than non-ablated tissue.

20. A method for visualizing ablated tissue, comprising:
advancing a catheter to a tissue in need of ablation, the catheter comprising an optically-enabled, steerable magnetic catheter body having a proximal end, a distal end, and one or more lumens therebetween, the catheter body having regions of differing flexibility along a length thereof; and one or more magnetic bodies positioned along a length of the catheter body and being responsive to an applied magnetic field, at least one of the one or more magnetic bodies located at or near the distal end of the catheter body so that the distal end of the catheter can be manipulated with a magnetic field;
directing the light from the distal end of the catheter body to excite nicotinamide adenine dinucleotide hydrogen (NADH) in an area of the tissue including ablated tissue and non-ablated tissue;
imaging the area of the tissue to detect NADH fluorescence of the area of the tissue; and
producing a display of the imaged, illuminated tissue, the display illustrating the ablated tissue as having less fluorescence than non-ablated tissue.
